(19) United States
(12) Patent Application Publication
(10) Pub. No.: US 2007/0020306 A1
(43) Pub. Date: Jan. 25, 2007

(54) Endovascular implant with an at least sectional active coating made of radjadone and/or a ratjadone derivative

(76) Inventor: Heinz-Peter Schultheiss, BERLIN (DE)

Correspondence Address: BROWDY AND NEIMARK, P.L.L.C.;624 NINTH STREET, NW, SUITE 300, WASHINGTON, DC, 20001-5303, US

(21) Appl. No.: 10/548,190

(22) PCT Filed: Mar. 12, 2004
(86) PCT No.: PCT/EP04/02621

§ 371 Date: Aug. 22, 2006

(30) Foreign Application Priority Data
Mar. 18, 2003 (DE) 103 11 729.6

(51) Int. Cl. A61K 31/366; A61F 2/02

(52) U.S. Cl. 424/423; 514/460

(57) ABSTRACT

An endovascular implant, comprising an at least sectional active coating (8) in which a (re)stenosis-inhibiting substance (10) based on a ratjadone derivative is embedded.

The invention relates to an endovascular implant having an at least sectional active coating of Ratjadone and/or a Ratjadone derivative, use of the substances for preparation of a drug for inhibiting restenosis, and a formulation with said substances.

Regarding the background of the invention, it can be stated that coronary heart disease is one of the leading causes of death in Western Europe and North America. According to the latest knowledge inflammatory processes, in particular, are the driving force behind arteriosclerosis. The process is presumably initiated by increased deposits of low-density lipoproteins in the intima of the vessel wall. After penetrating into the intima, the LDL particles are chemically modified by oxidants. The modified LDL particles, in turn, cause the endothelial cells, which line the inner vessel walls, to activate the immune system. Monocytes then enter into the intima and grow into macrophages. In the interaction with the also entering T cells, inflammatory mediators, such as immune messengers and substances with proliferating action, are released and the macrophages start to absorb the modified LDL particles. The forming lipid lesions of T cells and LDL particle-filled macrophages, which are called foam cells because of their appearance, represent an early stage of arteriosclerotic plaque. The inflammatory reaction in the intima, through corresponding inflammatory mediators, causes smooth muscle cells of the media of the vessel wall that are located further out, to migrate to a point under the endothelial cells. There they multiply and form a fibrous cover layer from the fiber protein collagen that separates the lipid core of foam cells located under it from the bloodstream. The profound structural changes that are then present in the vessel wall are collectively referred to as plaque.

Arteriosclerotic plaque initially expands only to a relatively small degree in the direction of the bloodstream, since the latter can expand to compensate for it. Over time, however, a narrowing of the blood channel occurs (stenosis), the first signs of which appear during physical exertion. The narrowed artery can then no longer adequately expand to increase the blood flow to the tissue it supplies. If a coronary artery is affected, the patient will often complain of a sensation of pressure and tightness behind the breastbone (angina pectoris). In the case of other arteries, painful cramps frequently are an indication of stenosis.

Stenosis can ultimately lead to a complete blockage of the blood stream (heart attack, stroke). According to recent studies, this occurs through plaque formation alone in approximately 15 percent of the cases. Additionally the gradual reduction of the fibrous cover layer of collagen that is caused by certain inflammatory mediators from the foam cells appears to be an important added factor. If the fibrous cover layer tears open, the lipid core can come into direct contact with the blood. Since tissue factors (TF) are also produced in the foam cells at the same time, as a result of the inflammatory reaction, which are very potent triggers of the clotting cascade, the forming blood clot can block the blood vessel.

Non-surgical methods for the treatment of stenosis have been in place for more than twenty years, whereby the blood vessel is re-expanded, among other methods, by means of balloon dilation (percutaneous transluminal coronary angioplasty, PTCA). The widening of the blood vessels, however, results in injuries to the vessel wall, which, even though they heal without problem, in up to 60% of the cases lead to proliferations due to the triggered cell growth, which ultimately lead to a renewed blockage of the vessel (restenosis). The widening also does not remove the physiological causes for the stenosis, i.e., the changes in the vessel wall. An additional cause of restenosis is the elasticity of the stretched blood vessel. After the removal of the balloon the vessel constricts excessively so that the vessel cross section is reduced (obstruction). The latter effect can be prevented only through placement of a stent. While it is true that an optimal vessel cross section can be achieved by using a stent, the use of stents also leads to tiniest injuries, which induce proliferation and can thus ultimately trigger restenosis.

By now comprehensive knowledge exists regarding the cell-biological mechanism and triggering factors for stenosis and restenosis. Restenosis—as has already been explained—occurs as a reaction of the vessel wall to the stretching of the arteriosclerotic plaque. Complex mechanisms of action induce the lumen-oriented migration and proliferation of the smooth muscle cells of the media and adventitia (neointimal hyperplasia). Under the action of various growth factors the smooth muscle cells produce a cover layer of matrix proteins (elasticin, collagen, proteoglycans), whose uncontrolled growth can gradually lead to a narrowing of the lumen. Systematic drug therapy provides for the administration of calcium antagonists, ACE inhibitors, anticoagulants, antiaggregants, fish oils, antiproliferative substances, anti-inflammatory substances and serotonin antagonists, for example, however significant reductions in restenosis rates have not been achieved by this method up to now.

The so-called concept of local drug delivery (LDD) now calls for the active substance or substances to be released directly at the location of the action, and limited to this area. For this purpose, a surface of the endovascular implant, i.e., especially that of a stunt, that is facing the blood vessel is provided with an active coating. The active component of the coating in the form of a therapeutic agent can be bound directly on the surface of the implant or embedded in a suitable drug carrier. In the latter case the active substance is released by means of diffusion and optionally gradual degradation of the biodegradable carrier.

Numerous preparations have been proposed as active substances and active-substance combinations, however, the effect demonstrated in therapeutic experiments up to know is only modest.

The invention is based on the object of further improving both the treatment of stenoses, as well as the prevention of restenoses, and making available in this context a particularly suitable endovascular implant with an active coating.

This object is met with an endovascular implant [¹] an at least sectional active coating, wherein the active coating includes a (re)stenosis-inhibiting substance of the following formula:
¹ Translator's note: It appears that the word “mit” (with) is missing in the German-language sentence.

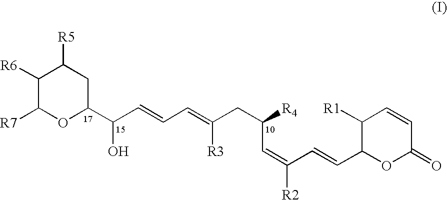

wherein R1, R2 and R3 are selected independently from one another from the group H, CH₃, and C₂H₅,

R4 is CH₃ or C₂H₅,

R5 is H or OH, and

R6 and R7 are selected independently from one another from the group H, CH₃, C₂H₅, n-C₃H₇, iso-C₃H₇, vinyl, CHCHCH₃ and C(CH₃)CH₂.

The active coating therefore contains as (re)stenosis-inhibiting substance the agent Ratjadone, which has been known for some time in principle as an antibiotic, antitumoral or cell-growth inhibiting compound. Reference is made in this context to DE 196 36 721 A1 and DE 101 06 647. The latter printed publication in particular also gives the general synthesis scheme for the Ratjadone derivatives.

Surprisingly it has now been discovered that the natural substance Ratjadone and Ratjadone derivatives inhibit the growth of aortal, smooth vascular muscle cells in the human. This effect already occurs starting at 1 to 100 nM, preferably 5 to 50 nM, so that already extremely low local concentrations are sufficient for an effective inhibition of restenosis in the region of an implant. This virtually precludes broader side-effects.

A Ratjadone derivative used as an active substance will preferably be the (+)-Ratjadone. The natural substance has proven particularly potent in first experiments, i.e., pharmacologically active even in the smallest concentrations of active substance. Preferred variants additionally provide for the C10 and C17 carbon atom to be R-configured if the C16 carbon atom is R-configured and at the same time neither R5 nor R6 nor R7 are H in above Formula I. Otherwise these radicals R5, R6 and R7 may also be H. Said derivatives are characterized by a potentially improved tolerance as compared to the natural substance.

According to a preferred variant of the invention, Ratjadone and its derivatives are embedded into a drug carrier. This allows for a simplification of the production of the coated implants and controlled release of the drug substance. Additionally an undesired flaking-off of the active substance during the implantation process, particularly during dilation of the stent, can be suppressed effectively. It goes without saying, of course, that the drug carrier must be biocompatible. The drug carrier is preferably additionally also biodegradable, so that a targeted dosing of the drug substance is possible via its degradation behavior. The use of glycosamino-glycans, especially hyaluronic acid, or of derivatives of these substances, has proven particularly advantageous in this context.

Glycosaminoglycans are negatively charged polysaccharides that consist of 1,4-linked disaccharide units. One component of this unit is a uronic acid (e.g., D-glucoronic acid, L-iduronic acid) that is linked via a β-(1→3) bond to an amino sugar.

A layer-thickness of the active coating in the case of drug carriers with embedded active substance is preferably between 3 and 30 μm, particularly between 8 and 15 μm. A weight mass per implant, i.e., the weight of the drug carrier plus active substance, is preferably in the range of 0.3 to 2 mg, particularly 0.5 to 1 mg. With these selected ranges, a high degree of local effect can be achieved without the dreaded side-effects being able to occur in the kidney, gall bladder, etc. Thin coatings of this type also do not tend to crack and accordingly resist a flaking-off when mechanically stressed.

If a biodegradable drug carrier is used, the elution characteristic can be influenced particularly by varying the cross-linking density of the polymer matrix or by varying the degree of polymerization. In addition to the degradation of the carrier, diffusion processes are important for the elution of the active substance. Structural properties of the carrier and active substance influence the diffusion speed in addition to many other factors.

Between the active coating and a main body of the implant, a passive coating may preferably be provided that contains amorphous silicone carbide. This allows for an improved adhesion of the active coating to the surface of the implant. Additionally, the passive coating by itself also already reduces the neointimal proliferation.

It is furthermore advantageous if a main body of the implant is formed of at least one metal or at least one metal alloy. It is additionally advantageous if the metal or the metal alloy is at least partly biodegradable. The biodegradable metal alloy may be especially a magnesium alloy. The stent according to the biodegradable variant is completely degraded over time, with the result that possible causes for an inflammatory and proliferative reaction of the surrounding tissue disappear as well.

The invention furthermore relates to a formulation for (re)stenosis inhibition that has a concentration of a Ratjadone substance according to one or more of claims 1 through 4 sufficient to inhibit (re)stenosis, and a pharmaceutically acceptable carrier.

Additional characteristics, details and advantages of the invention will become apparent from the following description, in which an example embodiment will be explained in more detail based on the appended drawings, in which

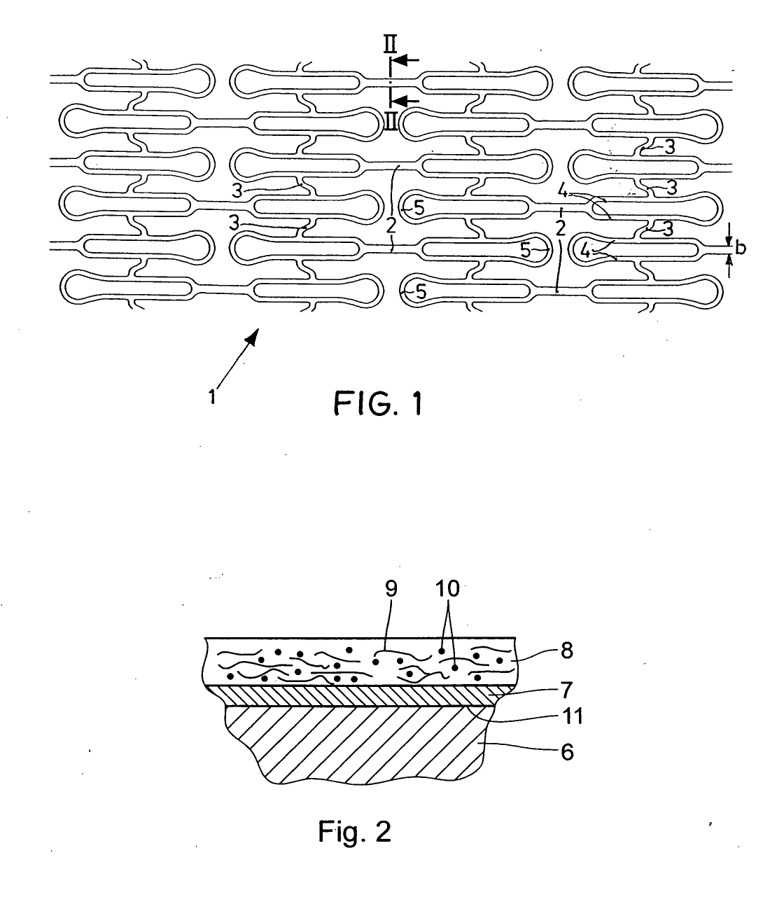

FIG. 1 shows a top view of an endovascular implant in the form of a stent, which is depicted unwound,

FIG. 2 shows an enlarged detail section through the implant according to the section line II-II of FIG. 1

As becomes apparent from FIG. 1, a dilatable stent 1 consists of a finely structured net of longitudinal links 2 and cross-links 3 connecting the former. The longitudinal links 2 branch out into strands 4 that are parallel to one another and are connected at the end in pairs, in each case, by an arc 5. On the left and right, relative to FIG. 1, the longitudinal links 2 extend with their branched-out strands 4 to the end of the overall tubular stent 1. In the direction of the cross links 3, the structure is curved cylindrically so that the cross links 3 that terminate at the top, relative to FIG. 1, transition into the cross links 3 that terminate at the bottom. Regarding their dimensions, the widths b of the links 2, 3, are in the sub-millimeter range.

From FIG. 2 the layer design of the structure of stent 1 is apparent. A main body 6, which may be formed of metal or a metal alloy, serves as the carrying element.

If the entire stent 1 is to be biodegradable, the main body 6 may be produced especially based on a biodegradable metal or a biodegradable metal alloy. Particularly suitable is a biodegradable magnesium alloy. Materials of this type have already been described sufficiently in prior art documents, so that a separate description may be dispensed with. Reference is made in this context particularly to the disclosure of DE 198 56 983 A1 of the applicant's.

Applied on this main body 6 is a passive coating 7, which will be explained in more detail below, and on it, in turn, an active coating 8 consisting of a drug carrier 9 and embedded therein a restenosis-inhibiting substance 10. The latter is symbolized in FIG. 2 by a dots.

The passive coating 7 provides for a particularly high degree of adhesion of the active coating 8 on the surface 11 of the main body 6 of the stent. The passive coating 7 is composed of amorphous silicon carbide. The production of structures of this type is known from the prior art, especially from patent document DE 44 29 380 C1 of the applicant's. Reference is made to the full disclosure of that printed publication, so that more detailed explanations regarding the production of the passive coating 7 will not be necessary at this point.

The above drug carrier 9 in the active coating 8 is formed by hyaluronic acid, which is biocompatible and permits a controlled release of the active substance 10 embedded therein. The drug carrier 9 additionally serves to prevent a flaking-off of the active coating 8 during the dilation or insertion of the stent 1 into an arterial vessel. To this end, the design of the stent should be adapted in such a way that the largest possible surface-contact exists to the vessel wall. This enhances an even elution of the active substance, which, as studies have shown, is substantially diffusion-controlled. Regions of high mechanical deformability will preferably be kept free of coating 7, 8 since there is an increased risk of the coating 7, 8 flaking off in these areas. Alternatively or to complement the design, the design of the stent may be specified such that when mechanical stress occurs, i.e., as a rule during the dilation of the stent, the occurring forces are distributed as evenly as possible across the entire stent surface. In this manner local over-stresses and ensuing cracking or even flaking-off of the coating can be prevented.

The actual active substance 10 in the drug carrier 9 in this specific example embodiment is formed by a Ratjadone derivative of the following formula: 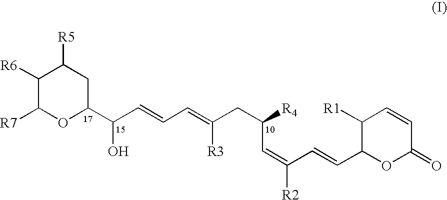
wherein R1, R2 and R3 are selected independently from one another from the group H, CH₃, and C₂H₅,

R4 is CH₃ or C₂H₅,

R5 is H or OH, and

R6 and R7 are selected independently from one another from the group H, CH₃, C₂H₅, n-C₃H₇, iso-C₃H₇, vinyl, CHCHCH₃ and C(CH₃)CH₂.

In this specific example, the C10, C17 and C16 carbon atoms are R-configured and the radicals R5, R6, and R7 are not occupied by hydrogen H.

If the drug carrier 9 is biodegradable, the elution characteristic of the active substance can be influenced by varying the cross-linking density of the polymer matrix or varying the degree of polymerization. This process presents itself especially for the above-mentioned drug carrier hyaluronic acid or polylactide. With an increasing cross-linking density and increasing molecular mass of the polymer, the amount of time generally increases as well, over which the active substance is released. The elution characteristic of an active coating of this type is preferably adjusted such that 10 to 30%, especially 15 to 25% of the active substance is released within the first two days.

The remainder of the remaining active substance should be released—also controlled via diffusion and degradation processes—successively within the first few months. It has been found, surprisingly, that these actually rather short periods of time already permit an effective suppression of neointimal proliferation.

The active coating 8 may additionally be structured in its design. For example, a lower cross-linking density may be provided in the outer regions of the active coating 8 than in the further inwardly situated regions. In this manner the degradation of the active coating 8 can initially occur faster after the implantation and, with an evenly distributed active substance concentration in the active coating 8, an altogether higher starting dose can be released than during the remaining period. Alternatively or to complement the design, this effect may also be achieved by specifying locally varying concentrations of the active substance 10 in the active coating 8 in such a way, for example, that the uppermost regions of the coating 8 have higher concentrations of the active substance. The active coating 8 is produced with the aid of a rotation diffuser, which creates a mist of micro-fine particles. Alternatively, ultrasound diffusers may be used as well. The coating takes place in steps in numerous cycles consisting of a wetting step of the stent in the generated spray mist and subsequent drying step of the precipitation on the stent by blowing off the excess. The multi-step production process allows for the creation of any desired layer thicknesses and—if desired—concentration gradients of the active substance or substances in individual layers of the active coating 8. If desired, multi-layered systems—for example for the combination of Ratjadone and Ratjadone derivatives—may be created in this manner as well, which are deposited one after the other.

A sterilization of the stent takes place by means of electron bombardment, and a partial cracking of the polymer chains of an optionally present polymeric carrier can be accepted in the case of high molecular weights of the polymer. The kinetic energy of the electrons is in the range of approximately 4 to 5 MeV, since an adequate sterilization is still ensured at these values with only minor penetration depth. The dosage is in the range between 15 to 35 kGy per stent. Studies have shown that only a minimal or no reduction in the biological activity of the active substances is caused by the sterilization method.

The generated layer thicknesses of the active coating 8 are generally in the range of 5 to 30 μm. Particularly advantageous are layer thicknesses in the range of 8 to 15 μm, since this ensures an essentially complete coverage of the surface 11 of the stent 1 at which structural problems, such as cracking and the like, do not yet need to be anticipated. Altogether approximately 0.3 to 2 mg, especially 0.5 to 1 mg, of coating material are applied if the active coating 8 contains a drug carrier.

To inhibit restenosis, the coating contains a sufficient concentration of Ratjadone and/or of a Ratjadone derivative. The elution characteristic is specified in the above manner such that the concentration of the substance(s) in the immediate vicinity of the coating is approximately 1 to 100 nM, especially 5 to 50 nM. In studies it has been demonstrated that these low concentration ranges already have a restenosis-inhibiting effect.

1. An endovascular implant having an at least sectional active coating, wherein the active coating contains a (re)stenosis-inhibiting substance of the following formula: 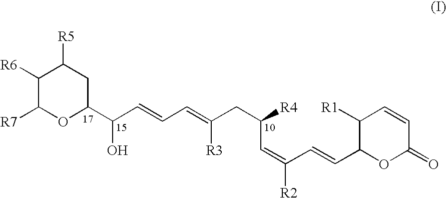
wherein R1, R2 and R3 are selected independently from one another from the group H, CH₃, and C₂H₅,
R4 is CH₃ or C₂H₅,
R5 is H or OH, and
R6 and R7 are selected independently from one another from the group H, CH₃, C₂H₅, n-C₃H₇, iso-C₃H₇, vinyl, CHCHCH₃ and C(CH₃)CH₂.

2. An implant according to claim 1, wherein the substance is (+)-Ratjadone.

3. An implant according to claim 1, wherein C10 and C17 are R-configured if C16 is R-configured and at the same time neither R5, nor R6, nor R7 are H.

4. An implant according to claim 1, wherein R5, R6 and R7 are H.

5. An implant according to claim 1, wherein the active coating (8) comprises a drug carrier (9) in which the active substance (10) is embedded.

6. An implant according to claim 5, wherein the drug carrier (9) is a glycosamino-glycan or derivatizing glycosamino-glycan.

7. An implant according to claim 6, wherein the glycosamino-glycan is hyaluronic acid or derivatizing hyaluronic acid.

8. An implant according to claim 1, wherein a layer thickness of the active coating (8) is 3 to 30 μm.

9. An implant according to claim 8, wherein the layer thickness of the active coating (8) is 8 to 15 μm.

10. An implant according to claim 5, wherein a total mass of the active coating (8) of drug carrier (9) and active substance (10) is 0.3 to 2 mg.

11. An implant according to claim 10, wherein the total mass of the active coating (8) of drug carrier (9) and active substance (10) is 0.5 to 1 mg.

12. An implant according to claim 5, wherein the drug carrier (9) is biodegradable.

13. An implant according to claim 1, wherein between the active coating (8) and a main body (6) of the implant a passive coating (7) is provided that contains amorphous silicon carbide.

14. An implant according to claim 1, wherein the main body (6) of the implant (1) is formed of at least one metal or at least one metal alloy.

15. An implant according to claim 14, wherein the metal or the metal alloy is at least partly biodegradable.

16. An implant according to claim 15, wherein the biodegradable metal alloy is a magnesium alloy.

17. (canceled)

18. A method for inhibiting restenosis in an endovascular implant procedure comprising implanting in a patient in need thereof an endovascular implant in accordance with claim 1.

19. A formulation for (re)stenosis inhibition, comprising
(a) a concentration of a (re)stenosis-inhibiting substance of the following formula: 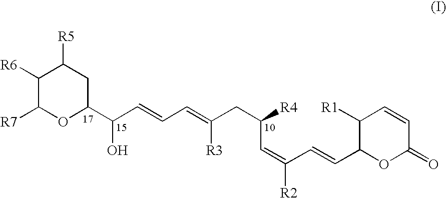
wherein R1, R2 and R3 are selected independently from one another from the group H, CH₃, and C₂H₅,
R4 is CH₃ or C₂H₅,
R5 is H or OH, and
R6 and R7 are selected independently from one another from the group H, CH₃, C₂H₅, n-C₃H₇, iso-C₃H₇, vinyl, CHCHCH₃ and C(CH₃)CH₂ and
(b) a pharmaceutically acceptable carrier.
